(19) United States
(12) Patent Application Publication
(10) Pub. No.: US 2025/0288304 A1
(43) Pub. Date: Sep. 18, 2025

(54) PHYSICIAN CONTROLLED HEMOSTATIC CLIP WITH A SINGLE HAND OPERATED HANDLE

(71) Applicant: COOK MEDICAL TECHNOLOGIES LLC, Bloomington, IN (US)

(72) Inventors: Hunter Pelham, Winston-Salem, NC (US); Michael J. Brecht, Pfafftown, NC (US); Meredith Lee, Winston-Salem, NC (US); Dustin Mullins, Winston-Salem, NC (US); Katarina McGarry, Virginia Beach, VA (US); Michelle Smith, Winston-Salem, NC (US); John Crowder Sigmon, Winston-Salem, NC (US); Mikayla Lamb, Winston-Salem, NC (US)

(21) Appl. No.: 19/003,291

(22) Filed: Dec. 27, 2024

Related U.S. Application Data
(60) Provisional application No. 63/615,663, filed on Dec. 28, 2023.

(51) Int. Cl. A61B 17/128; A61B 17/00; A61B 17/12

(52) U.S. Cl. CPC A61B 17/1285; A61B 2017/00296; A61B 2017/00336; A61B 2017/00818; A61B 2017/00876; A61B 2017/12004

(57) ABSTRACT

The present disclosure relates to a physician controlled single hand operated handle system. The system includes a handle. The handle includes an actuator, a stem disposed within the actuator and longitudinally movable within the actuator, a sheath attached to the stem, and an elongate member extending through the sheath. The elongate member is longitudinally constrained by the actuator and rotatably constrained by the stem.

### BACKGROUND

### 1. Technical Field text

The present disclosure relates to medical devices for the control of gastrointestinal bleeding. In particular, the present disclosure relates to a physician controlled hemostatic clip having a single hand operated handle.

### 2. Background Information

Conventionally, a clip may be introduced into a body cavity through an endoscope to grasp living tissue of the body cavity for hemostasis, marking, and/or ligating. Such clips are often known as surgical clips, endoscopic clips, hemostasis clips and vascular clips. In addition, clips are now being used in a number of applications related to gastrointestinal bleeding such as peptic ulcers, Mallory-Weiss tears, Dieulafoy's lesions, angiomas, post-papillotomy bleeding, and small varices with active bleeding. Clips have also been attempted for use in closing perforations in the stomach.

Gastrointestinal bleeding is a somewhat common and serious condition that is often fatal if left untreated. This problem has prompted the development of a number of endoscopic therapeutic approaches to achieve hemostasis such as the injection of sclerosing agents and contact thermo-coagulation techniques. Although such approaches are often effective, bleeding continues for many patients and corrective surgery therefore becomes necessary. Because surgery is an invasive technique that is associated with a high morbidity rate and many other undesirable side effects, there exists a need for highly effective, less invasive procedures.

Mechanical hemostatic devices such as clips have been used in various parts of the body, including gastrointestinal applications. Because gastrointestinal bleeding is an emergency situation in which accurate and rapid application of a hemo-clip is required. Generally, a physician relies on support staff to assist in the orientation to obtain the desired position of the device. Insufficient communication may result in an increase in the amount of time to obtain hemostasis. Hence, there is a need for the physician to have control over the rotation, opening/closing, and deployment of the hemo-clip without having to rely on support staff and to minimize potential miscommunication.

### SUMMARY

According to various aspects, the present disclosure relates to a physician controlled single hand operated handle system. The system includes a handle. The handle includes an actuator, a stem disposed within the actuator and longitudinally movable within the actuator, a sheath attached to the stem, and an elongate member extending through the sheath. The elongate member is longitudinally constrained by the actuator and rotatably constrained by the stem.

In embodiments of the above, the stem includes a proximal end, and the stem includes a wheel at the proximal end, wherein the wheel is rotatable with the stem.

In any of the above embodiments, the elongate member is held in an insert, and the insert is longitudinally constrained in a cutout in the actuator. In further embodiments, the elongate member is held in the insert with a set screw. In additional further embodiments, the insert is rotatably constrained by the stem, wherein the elongate member and stem are rotatable relative to the actuator. In additional further or alternative embodiments, the insert is rotatably constrained by the actuator.

In any of the above embodiments, the sheath is connected to a sheath grip and the sheath grip holds the sheath relative to the stem. In further embodiments, the sheath grip is non-rotatably retained by a cap in a rigid mount and the elongate member and sheath pass through the rigid mount. In yet further embodiments, the cap is non-rotatably secured to the stem. In additional further embodiments, a slider extends through a slot defined in the rigid mount, wherein the slider is connected to the cap and the slider is configured to move the cap longitudinally in a rigid mount. In additional embodiments, the sheath grip is non-rotatably retained by a cap and the cap is connected to an accordion and the elongate member and sheath pass through the accordion. In yet further embodiments, the accordion includes a scope mount at a distal end of the handle.

In any of the above embodiments, a distal end of the handle comprises a scope mount configured to engage with a mount of an endoscope. In further embodiments, the scope mount includes a first magnet received in a first channel defined in the scope mount and the system further comprising a port mount magnetically couple-able to the scope mount, wherein the port mount includes a second magnet received in a second channel defined in the port mount. In yet further embodiments, the port mount includes a seal.

In any of the above embodiments, the elongate member has a proximal end and a distal end, and wherein the proximal end is operatively coupled to the actuator and the distal end is operatively coupled to a clip. In further embodiments, the clip exhibits an open configuration and a closed configuration wherein proximal longitudinal movement of the stem relative to the actuator opens the clip, and wherein a first distal longitudinal movement of the stem relative to the actuator closes the clip. In yet further embodiments, a second distal longitudinal movement of the stem relative to the actuator releases the clip from the system. In additional further embodiments, the clip includes a pair of jaws and the system further comprises a clip housing configured to receive the pair of jaws.

In alternative embodiments, the elongate member is at least one of a needle and a trocar.

In further aspects, the present disclosure relates to a physician controlled single hand operated handle system. The system includes a handle with a proximal end, a distal end, an actuator including a gripping portion, an elongate stem disposed within the gripping portion that is rotationally and longitudinally movable within the gripping portion. A thumb wheel is attached to a proximal end of the stem and is rotationally and longitudinally moveable with the stem. The thumb wheel is configured to be engaged with the thumb of the hand of the operator at the same time the hand of the operator grips the gripping portion of the actuator. The system further includes a sheath attached to the stem, a drive wire or other elongate member extending through the sheath; and a clip operatively coupled to the drive wire. Rotation of the thumb wheel rotates the clip. When the sheath and the stem/cap move proximally, the drive wire remains stationary and the clip opens. When the sheath and the stem/cap move distally, the drive wire remains stationary and the clip closes.

The drive wire has a proximal end and a distal end. The proximal end is operatively coupled to the gripping portion and the distal end is operatively coupled to the clip. The clip has an open configuration and a closed configuration where proximal longitudinal movement of the stem relative to the actuator opens the-clip, and where a first distal longitudinal movement of the stem relative to the actuator closes the clip. A second distal longitudinal movement of the stem relative to the gripping portion releases the clip from the system.

### BRIEF DESCRIPTION OF THE DRAWINGS

The accompanying drawings incorporated in and forming a part of the specification illustrate several aspects of the present disclosure, and together with the description serve to explain the principles of the disclosure. In the drawings:

FIG. 1 is a partial perspective view of an exemplary hemo-clip for use with the handles of the present disclosure;

FIG. 2 shows the distal end of the exemplary hemo-clip system of FIG. 1 with the clip in a closed position according to embodiments of the present disclosure;

FIG. 3 shows the distal end of the exemplary hemo-clip system of FIG. 1 with the clip in an open position according to embodiments of the present disclosure;

FIG. 4 shows an embodiment of a novel single hand operated handle for use with the hemo-clip system of FIG. 1;

FIG. 5 shows the novel single hand operated handle of FIG. 4 with the grip feature in a first position;

FIG. 6 shows the novel single hand operated handle of FIG. 4 with the grip feature in a second position;

FIG. 7 shows an embodiment of a novel single hand operated handle;

FIG. 8 shows the novel single hand operated handle of FIG. 7;

FIG. 9 shows an embodiment of a novel single hand operated handle mounted on an accessory port of an endoscope;

FIG. 10 shows the novel single hand operated handle of FIG. 9;

FIG. 11 shows an embodiment of a novel single hand operated handle including a scope mount and a port mount;

FIG. 12 shows a cross-section of the novel single hand operated handle of FIG. 11;

FIG. 13 shows a port mount for use with the scope mount according to embodiments of the present disclosure;

FIG. 14 shows an embodiment of a novel single hand operated handle mounted on an accessory port of a scope;

FIG. 15 shows the novel single hand operated handle of FIG. 14;

FIG. 16 shows the novel single hand operated handle of FIG. 14;

FIG. 17 shows the novel single hand operated handle of FIG. 14;

FIG. 18 shows the novel single hand operated handle of FIG. 14;

FIG. 19 shows the inner mechanism of the first and second embodiments;

FIG. 20 shows a close up of the inner mechanism of the first and second embodiments;

FIG. 21 shows the inner mechanism of the third embodiment;

FIG. 22 shows the inner mechanism of the fourth embodiment;

FIG. 23 shows an exploded view of the first and second embodiments shown in FIGS. 19 and 20;

FIG. 24 shows a rigid guide according to embodiments of the present disclosure;

FIG. 25 shows a stem according to embodiments of the present disclosure;

FIG. 26 shows an actuator according to embodiments of the present disclosure;

FIG. 27 shows an insert according to embodiments of the present disclosure;

FIG. 28 shows a cap according to embodiments of the present disclosure;

FIG. 29 shows sliders according to embodiments of the present disclosure;

FIG. 30 shows sliders according to embodiments of the present disclosure;

FIG. 31 shows an insert according to embodiments of the present disclosure;

FIG. 32 shows advance/retract sliders screwed into a cap according to embodiments of the present disclosure;

FIG. 33 shows advance/retract sliders with a sliding element according to embodiments of the present disclosure;

FIG. 34 shows a side view of the clip end of the system according to embodiments of the present disclosure;

FIG. 35 shows a partial perspective/side view of the clip end according to embodiments of the present disclosure;

FIG. 36 shows another partial perspective/side view of the clip end according to embodiments of the present disclosure;

FIG. 37 shows a side view of the clip end according to embodiments of the present disclosure;

FIG. 38 shows a cross-section of the clip end of the system of FIGS. 30 through 33;

FIG. 39 shows a cross-section of the clip end of the system of FIGS. 30 through 33;

FIG. 40 shows a perspective cross-section of the clip end according to embodiments of the present disclosure;

FIG. 41 is a perspective view of the connection between the driver, the driver/drive wire connector and the drive wire;

FIG. 42 is another view of the connection between the driver, the driver/drive wire connector and the drive wire;

FIG. 43 is a perspective view of the clip in the open configuration;

FIG. 44 is a top view of the driver;

FIG. 45 is an enlargement of inset A of FIG. 44;

FIG. 46 is a further embodiment wherein the drive wire and clip are replaced by a needle;

FIG. 47 is yet a further embodiment wherein the clip is replaced by forceps;

FIG. 48 illustrates a cross-section of the forceps and sheath with the forceps in an open position; and

FIG. 49 illustrates a cross-section of the forceps and sheath with the forceps in a closed position.

### DETAILED DESCRIPTION

The terms “proximal” and “distal” as used herein are intended to have a reference point relative to the user. Specifically, throughout the specification, the terms “distal” and “distally” shall denote a position, direction, or orientation that is generally away from the user, and the terms “proximal” and “proximally” shall denote a position, direction, or orientation that is generally towards the user. For example, the handle is at the proximal end and the clip is at the distal end. In this particular application, distal refers to the clip end of the system, whereas proximal refers to the handle end of the system.

The present disclosure relates to medical devices for the control of gastrointestinal bleeding. In particular, the present disclosure relates to a physician controlled hemostatic clip having a single hand operated handle. Generally, the hemostatic clip includes a housing, first and second jaws rotatable relative to the housing, a driver, and an elongate drive wire, which may be disconnected from the driver, first and second jaws, and the housing which are left in vivo engaged with the tissue. Suitable devices are described in U.S. Pat. No. 9,339,270, “Medical Devices With Detachable Pivotable Jaws,” U.S. Pat. No. 8,771,293, “Medical Devices With Detachable Pivotable Jaws,” U.S. Pat. No. 8,939,997, “Medical Devices With Detachable Pivotable Jaws,” U.S. Pat. No. 8,858,588, “Medical Devices With Detachable Pivotable Jaws,” all of which are incorporated by reference herein in their entireties. Another clip suitable for use with the present disclosure is described in U.S. Pat. No. 11,576,682, “Medical Devices With Detachable Pivotable Jaws,” which is incorporated by reference in its entirety.

While the present technology is described primarily herein in connection with hemo-clips used in preventing or controlling gastrointestinal bleeding, the technology herein is not limited to these applications and may alternatively be employed in other applications including the introduction and actuation of needles, trocars, cannulas, snares, biopsy forceps and other devices during various medical procedures such as aspiration, tissue resection, injections guided by ultrasounds and other imaging techniques, or the delivery of therapeutic compositions.

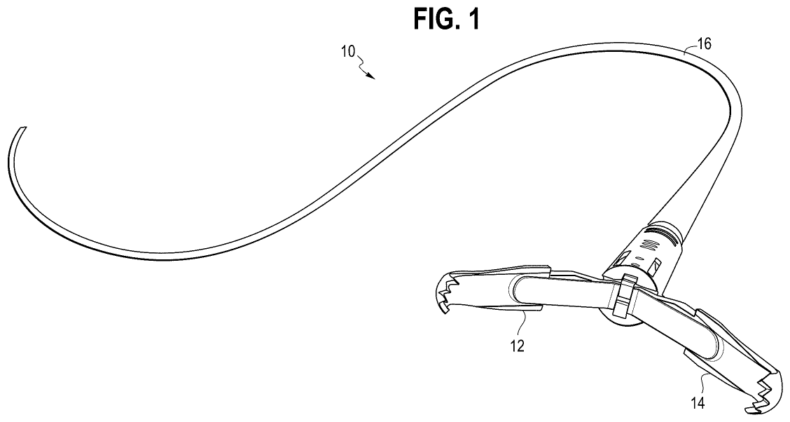

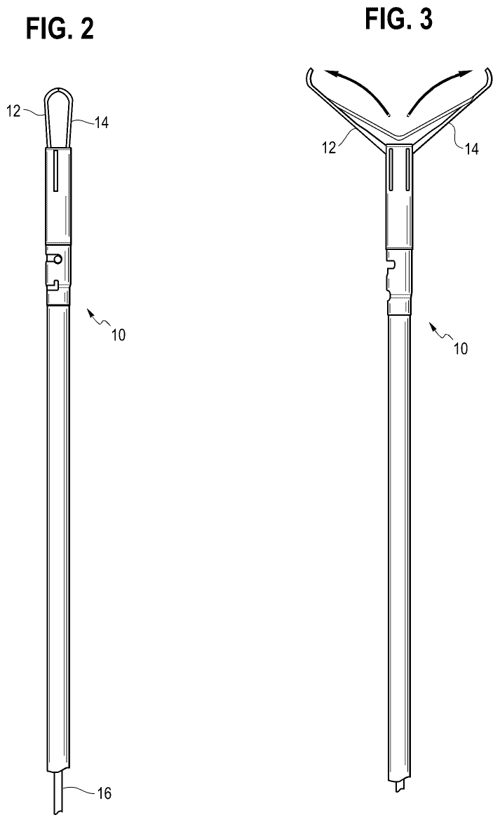

FIG. 1 is a partial perspective view of an exemplary hemo-clip system 10 for use with the handles of the present disclosure. The clip system 10 includes a pair of jaws 12, 14 which are connected to a drive wire 16 (shown in FIG. 2) via driver (not shown). FIG. 2 shows the distal end 18 of the exemplary hemo-clip of FIG. 1 with the pair of jaws 12, 14 in a closed position. FIG. 3 shows the distal end of an exemplary hemo-clip system of FIG. 1 with the pair of jaws 12, 14 in an open position.

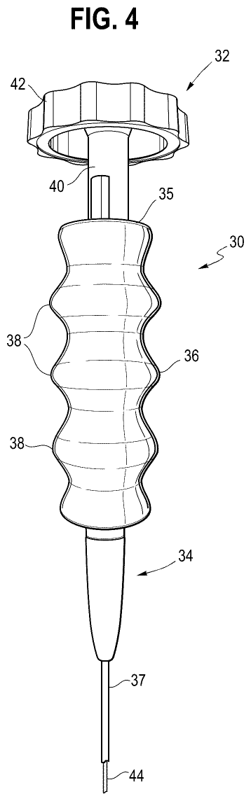

FIG. 4 shows a first embodiment of a novel single hand operated handle for use with the hemo-clip system of FIG. 1, or other devices, and having a rigid sheath. In this embodiment, the single hand operated handle 30 has a proximal end 32, a distal end 34, an actuator 36 including a grip feature, shown here as having gripping ridges 38. The actuator 36 constrains an internal retainer for an elongate member, such as a drive wire 44 that opens, closes, and deploys the clip when the stem 40 moves relative to the actuator 36. The handle 30 is mounted over the stem 40. The stem 40 is slidably and rotationally disposed through a channel 35 of the actuator 36. A wheel 42 is disposed at the proximal end of the stem 40. The actuator 36 constrains an internal retainer to the drive wire 44 and deploys the clip from its closed position to its open position when the stem 40 is moved relative to the actuator 36.

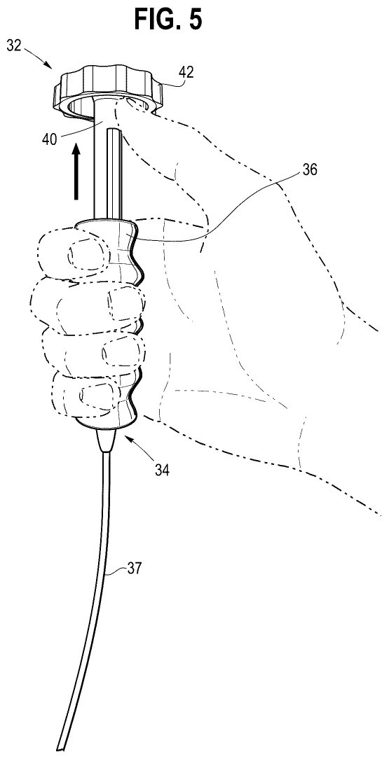

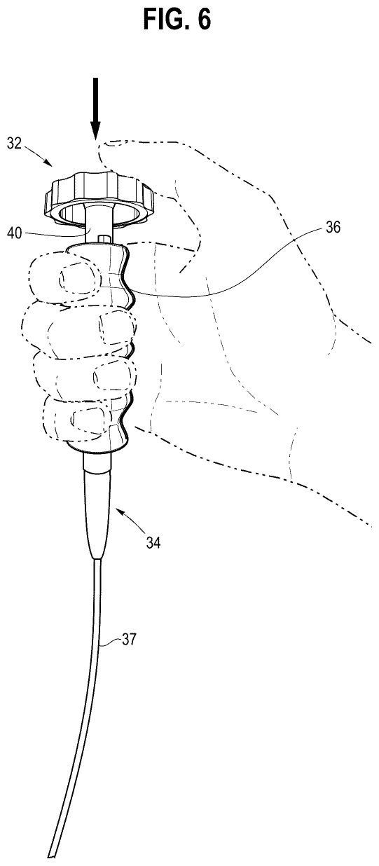

FIG. 5 shows the novel single hand operated handle of FIG. 4 with the stem 40 in a first position relative to the actuator 36, where the pair of jaws 12, 14 are in an open position as shown in FIG. 3. Opening the pair of jaws 12, 14 from the closed position to the open position is accomplished by moving the stem 40/wheel 42 proximally (as shown by the arrow in FIG. 5) away from the actuator 36. FIG. 6 shows the novel single hand operated handle of FIGS. 4 and 5 with the stem 40/wheel 42 in a second position relative to the actuator 36, where the pair of jaws 12, 14 are in a closed position as shown in FIG. 2. Closing the pair of jaws is accomplished by moving the stem 40/wheel 42 distally toward the actuator 36 (as indicated by the arrow). Deploying the clip is accomplished by further moving the stem 40/wheel 42 distally toward the actuator 36 to release (deploy) the clip from the system. The close function can be performed by squeezing the stem 40/wheel 42 and the actuator 36 together. The deployment function is also performed by squeezing the stem 40/wheel 42 and the actuator 36 together. The actuator 36 rotates freely relative to the stem to permit rotation of the clip at the distal end of the system. The wheel 42 is rotated relative to the actuator 36 in order to rotate the pair of jaws 12, 14. The drive wire 44 may be attached to the actuator 36 within the channel 35 of the actuator 36. Hence, when the stem/wheel 40, 42 are moved proximally relative to the actuator 36, sheath 37 (as explained in detail) below also moves proximally to open the clip. The drive wire 44 extends from the pair of jaws through the sheath to the handle. In this embodiment, the sheath has a variable thickness along its length from its proximal end at the handle to its distal end. Hence, the sheath will have variable stiffness/flexibility along its length, where it is flexible along its length with more rigidity or stiffness toward the handle to enable single handed use. This allows the distal end to be flexible and navigate the bends of the anatomy and the endoscope. With a stiffer proximal portion, the proximal end, including the handle, outside of the endoscope can support itself. In one example, about 20 cm of the proximal portion of the sheath are stiffer/more rigid. Operation of this handle requires only the action of the user with no input whatsoever required of support staff during the procedure.

In a second embodiment of the novel single hand operated handle, the handle has a rigid scope mount for mounting the system to the accessory channel of an endoscope or other device. This embodiment provides a rigid guide for the handle stem and maintains the position when the handle is released from the physician's grip. In other words, even when the physician moves his/her hand from the handle, the position of the handle is maintained on the endoscope.

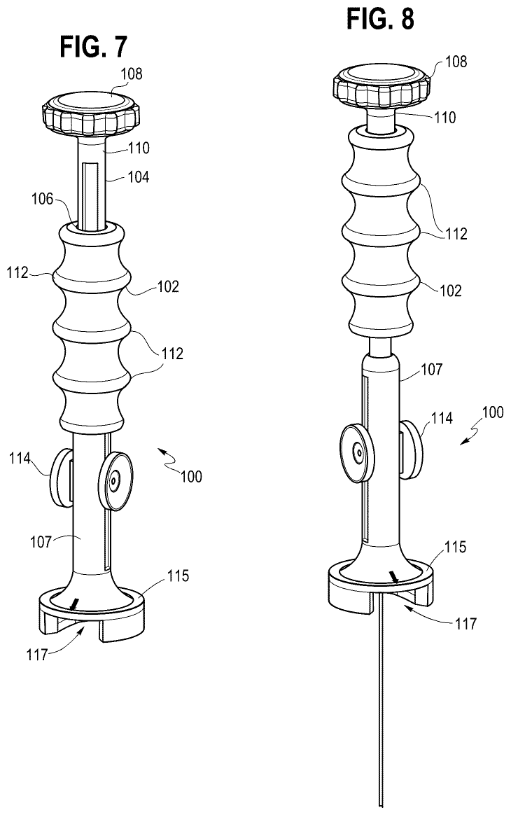

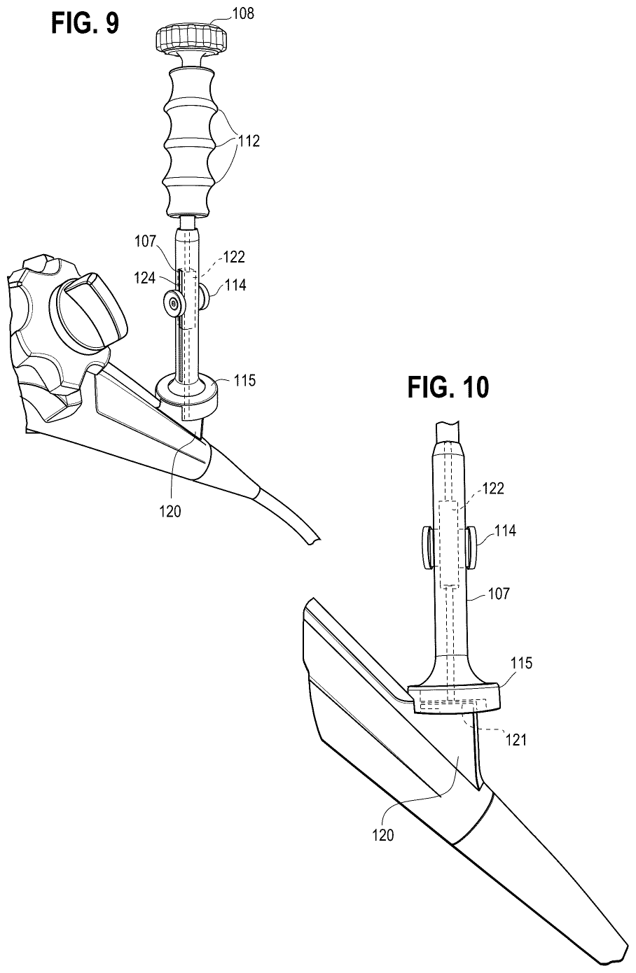

As shown in FIGS. 7 and 8, the second embodiment includes handle 100. The handle 100 includes an actuator 102, a stem 104 rotationally and slidably disposed within a channel 106 of the actuator 102, a rigid guide 107 through which the handle stem 104 extends, a rotatable wheel 108 at the proximal end 110 of the stem 104 configured to rotate the stem 104. The actuator 102 includes a grip feature having gripping ridges 112 for the user to grip. The actuator 102 has an internal retainer (not shown) for the drive wire 113 (not shown) that opens, closes, and deploys the clip when the actuator 102 is moved relative to the stem 104. The handle 100 further includes an advance/retract slide 114 disposed on the rigid guide 107 and a scope mount 115 at the distal end 116 of the handle configured to be mounted on an endoscopic device. The scope mount 115 features a channel 117 configured to mate with an endoscope 120 or other device as shown in FIGS. 9 and 10.

FIG. 7 shows the actuator 102 in a position distally away from the wheel 108 in which the pair of jaws of the clip is opened. FIG. 8 shows the actuator 102 in a position proximally toward the wheel 108 in which the pair of jaws of the clip is closed. In operation, the operator moves the wheel 108 proximally away from the actuator 102 to open the clip. The stem 104 and the wheel 108 rotate together to apply torque to the drive wire and rotate the clip at the system distal end. The actuator 102 rotates freely relative to the stem 104 and does not apply torque to the drive wire. Alternatively, the actuator 102 may apply torque to the stem 104 to rotate the clip at the distal end. To deploy (release) the clip, the actuator 102 and the wheel 108 are moved further closer together to overcome a deployment force threshold, thereby releasing the clip. Opening of the clip is accomplished by moving the actuator 102 and the wheel 108 away from each other as shown in FIG. 7. Closing the clip is accomplished by moving the actuator 102 and the wheel 108 closer to each other as shown in FIG. 8. In embodiments, the wheel 108 is grooved and assists with the rotation of the stem. A further alternative design may include a pivoting mount to adjust the angle of the mount relative to the endoscope to allow for ergonomic positioning. A seal may be incorporated into the mount to allow it to also function as a biopsy cap for the endoscope.

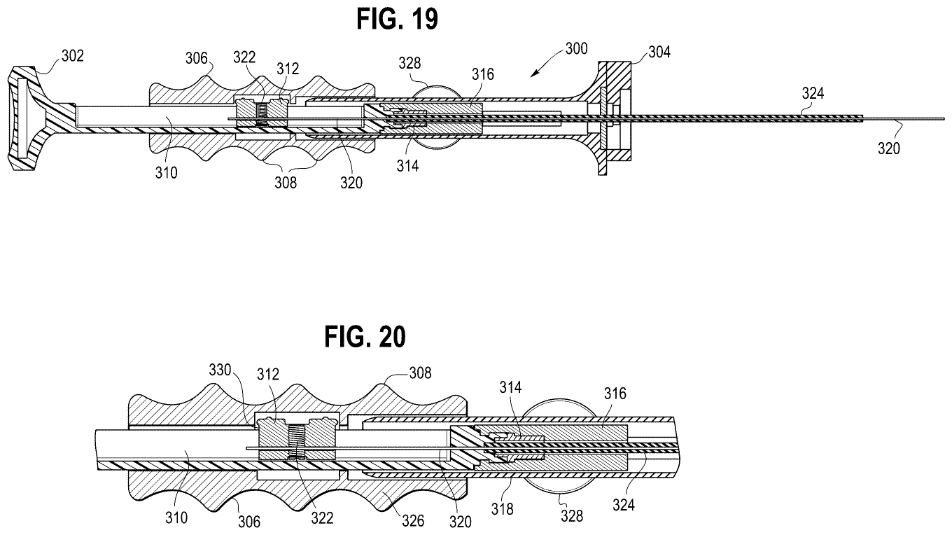

FIG. 9 shows the handle 100 of FIGS. 7 and 8 mounted on an endoscope 120 for deploying a clip within a patient. FIG. 10 shows a close up view of the mounting of the handle 100 of FIGS. 7 through 9 on the endoscope 120. As shown, the channel 117 is slid over the mount 121 of the endoscope. As shown in FIG. 9, the advance/retract slide 114 is disposed on and within the rigid guide 107. The slide portion 122 of the slide 114 is disposed within the lumen of the rigid guide 107 with a slider 114 disposed external to the rigid guide 107 to facilitate sliding of the slide portion 122 within the lumen of the rigid guide 107. The proximal end of the slide portion 122 is attached to the stem via cap 316 (as shown in FIG. 19). The slide 114 moves longitudinally within a longitudinal slot/linear track 124 in the rigid guide 107. When the slide 114 is pushed distally, the clip at the distal end of the system along with the sheath are advanced out of the distal end of the endoscope. When the slide 114 is pulled toward the proximal end, the clip and sheath are retracted back into the endoscope. Because the slide 114 is attached to the stem 104, when the slide moves either proximally or distally, the stem moves with it relative to the scope mount. The distal advancement of the slide 114 pushes the distal end of the device further out of the scope when activated. The slide 114 is on the linear track 124 so when the slide 114 is pushed distally, the clip is advanced out of the distal end of the endoscope. When the slide 114 is pulled to proximal end of the handle, the distal end of the clip is retracted back into the endoscope. The slide 114 is attached to the stem, thus, when the slide 114 is activated the entire stem moves with it relative to the scope mount.

As such, with the embodiment of FIGS. 7 through 10, the handle is immovably mounted to the scope and as such allows the clinician/physician to single handedly retract and advance the clip out of the scope, rotate the scope, and open, close and deploy the clip without any assistance.

In a third embodiment of the novel single hand operated handle, the handle has a rigid mount for releasably mounting the system to the accessory channel of an endoscope or other device. This embodiment provides a rigid guide for the handle stem and maintains its position when the handle is released from the physician's grip. In other words, even when the physician moves their hand from the handle, the position of the handle is maintained on the endoscope. This embodiment also provides a magnetic coupling between the handle and the accessory port of the endoscope allowing the physician to remove the system from the accessory channel while maintaining a seal over the accessory port of the endoscope.

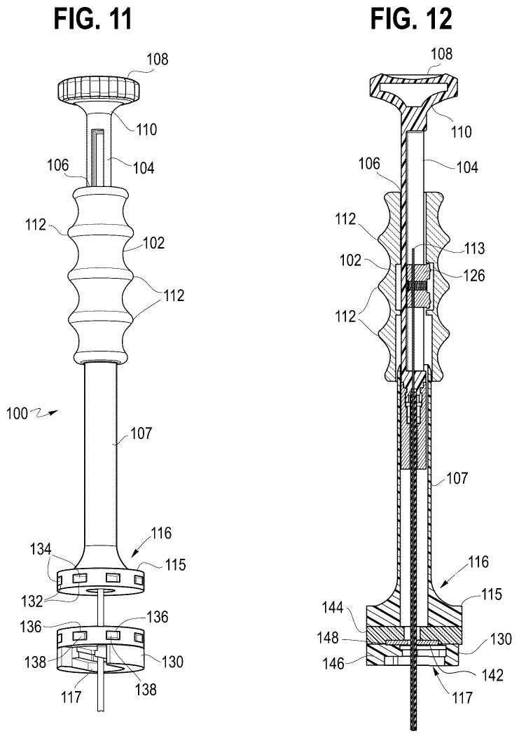

As shown in FIGS. 11 through 13, the second embodiment includes handle 100. Handle 100 includes an actuator 102 including a grip feature, a stem 104 rotationally and slidably disposed within a channel 106 of the actuator 102, a rigid guide 107 through which the handle stem 104 at least partially extends, a rotatable wheel 108 at the proximal end 110 of the stem 104 configured to assist in rotating the stem 104. The grip feature includes ridges 112 for the user to grip. The actuator 102 has an internal mount or insert 126 for the drive wire 113, which opens, closes, and deploys the clip when the actuator 102 is moved longitudinally relative to the stem 104. The handle 100 further includes a scope mount 115 at the distal end 116 of the handle configured to be mounted on an endoscopic device. FIG. 11 shows the actuator 102 in a position distally away from the wheel 108 in which the pair of jaws of the clip is opened.

FIG. 12 illustrates the scope mount 115 magnetically coupled to a port mount 130. The port mount 130 includes a channel 117 configured to mate with an endoscope port, such as an accessory port, of the endoscope. The scope mount 115 defines one or more channels 132 therein, such as around the periphery of the scope mount 115 as illustrated. One or magnets 134 are received in at least one of the channels 132. The port mount 130 also defines one or more channels 136 therein, such as around the periphery of the port mount 130 as illustrated and one or more magnets 138 are received in at least one of the channels 136.

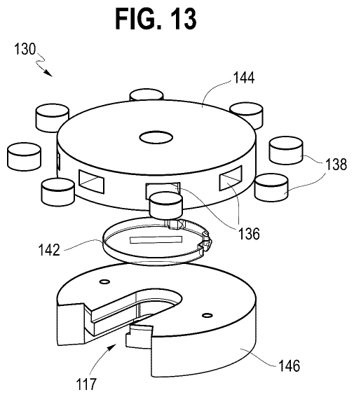

FIGS. 12 and 13 illustrate a seal 142 housed in the port mount 130. The seal 142 provides a hemostatic valve to prevent fluids or materials from entering or leaving the endoscope port once the handle 100 and system are removed. The seal 142 is sandwiched between an upper portion 144 of the port mount 130 and the lower portion 146 of the port mount 130. As illustrated, the upper portion 144 of the port mount 130 defines a recess 148 for receiving the seal 142. It should be appreciated that, alternatively, the lower portion 146 of the port mount 130, or both the upper portion 144 and the lower portion 146 of the port mount 130 may define a recess for receiving the seal 142. The upper portion 144 of the port mount 130 and the lower portion 146 of the port mount 130 are affixed together using at least one of an adhesive, screws, features that create an interference fit, and ultrasonic welding. In the illustrated embodiment, the upper portion 144 of the port mount 130 also includes the one or more channels 132 for receiving the magnets 134.

In the embodiment of FIGS. 11 through 13, the handle is mounted to the scope and as such allows the clinician/physician to single handedly retract and advance the clip out of the scope, rotate the scope, and open, close and deploy the clip without assistance. Further, the handle is releasable from the endoscope while maintaining a seal over the accessory port of the endoscope.

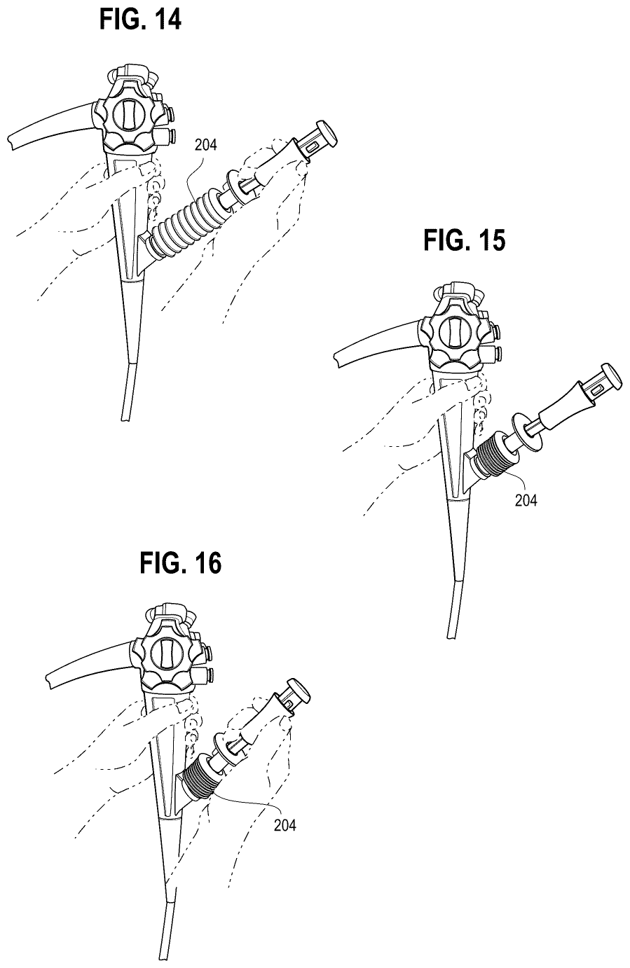

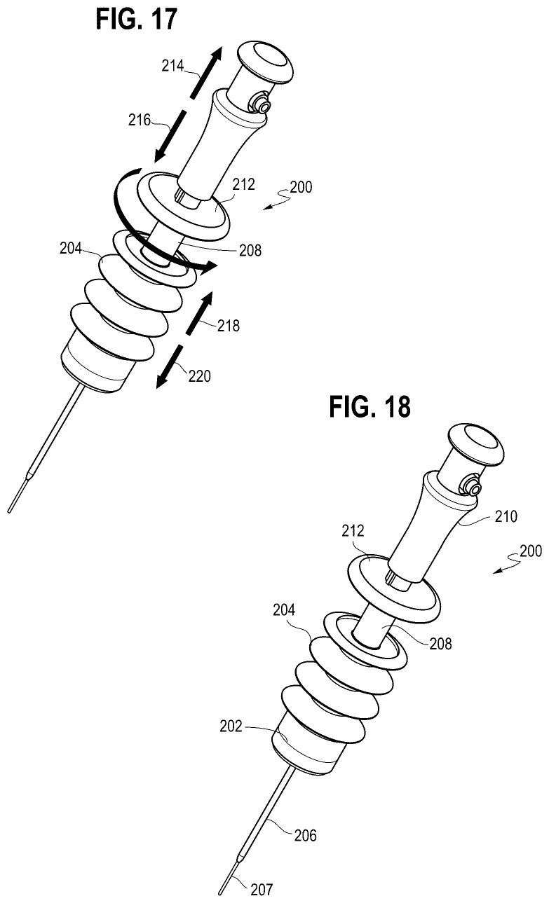

In a fourth embodiment, shown in FIGS. 14 through 18, a handle 200 includes a mount 202 that is mounted onto the accessory channel of the endoscope via an accordion feature 204. A sheath 206 runs through the accordion feature 204 which is configured to allow the clip to be retracted within or advanced out of the distal end of the endoscope by retracting/advancing the accordion feature 204. The drive wire 207 extends through the sheath 206. The accordion feature 204 is docked onto the scope using a snap feature or other suitable attachment. Like the bellows of an accordion, the accordion feature 204 is flexible, compressible and expandable. The flexibility of the accordion feature 204 allows the user to move the handle 200 off of the axis of the accessory channel to aid in comfort while driving the endoscope. The handle 200 includes a stem 208 that is attached onto the accordion feature 204 and an actuator 210 that slides over the stem 208. This allows for opening, closing, and deployment of the clip by movement of the actuator 210 along the stem 208. The stem 208 has a platform 212 for the user to rest their fingers in order to assist in the movement of the actuator 210. The handle can rotate the clip by rotating either the stem 208 or the actuator 210. An alternative design can have the stem 208 docked onto the scope using the concept of the embodiments described above. In another embodiment, the accordion 204 may be stiffer to retain position when dropped. A seal may be incorporated into the mount to allow it to function as a biopsy cap for the endoscope. As shown in FIG. 17, when the actuator 210 is moved distally, in the direction of arrow 216, the clip is opened. When the actuator 210 is moved proximally, as shown by arrow 214, the clip is closed. Once the clip is closed, further proximal movement of the actuator 210 releases the clip. When platform 212 is moved distally, in the direction of arrow 216, the entire system including the clip and sheath is moved out of the scope and when platform 212 is moved proximally, in the direction of arrow 214, the entire delivery system including the clip and sheath are moved into the scope.

FIGS. 19 and 20 show the internal mechanism of the first and second embodiments. As shown, the handle 300 includes a proximal end 302, a distal end 304, a grip feature 306 having gripping ridges 308, stem 310, insert 312 (which may be brass), sheath grip 314, cap 316, rigid guide 318, drive wire 320, set screw 322, coil spring/sheath 324, and an actuator 326. Sheath grip 314 holds sheath/coil spring 324 in place relative to the stem 310. Sheath grip 314 is retained in cap 316 in a non-rotatable manner. The cap 316 secures the sheath grip 314 to the stem 310 and may be either rotatable or non-rotatable relative to the stem 310. Cap 316 is also held in the rigid guide 318; however, the rigid guide 318 may not constrain rotational movement of the cap 316.

Set screw 322 holds the drive wire 320 in place relative to the insert 312 which secures the drive wire 320 inside the actuator 326. The insert 312 is constrained longitudinally in the actuator 326 by cutout 330. When a user moves the actuator 326 relative to the stem 310, this applies force to the drive wire 320 while the sheath 324 stays in place. This allows the clip (not shown) to open when the actuator 326 is moved distally to open and close when moved proximally. Cap 316 moves longitudinally forward and backward inside rigid guide 318. Sliders 328 are secured onto cap 316 allowing the user to move the entire assembly, advancing/retracting the clip and sheath in and out of the endoscope. Stem 310 can rotate relative to the cap 316 and thus the rigid guide 318. Alternatively, the stem 310 is non-rotatable relative to the cap 316. The stem 310 rotates the insert 312, i.e., the insert 312 is rotatably constrained by the stem 310, which translates this rotational force to the drive wire 320 while the sheath 324 remains in place. This allows the user to rotate the clip. Cutout 330 in the actuator 326 allows for the insert 312 and stem 310 to rotate to apply torque onto the drive wire 320 without rotating the actuator 326. This allows the user to maintain their grip on the actuator 326 while rotating the drive wire 320 and the clip. This also allows for rotation of the actuator 326 without causing the insert 312 and drive wire 320 to rotate.

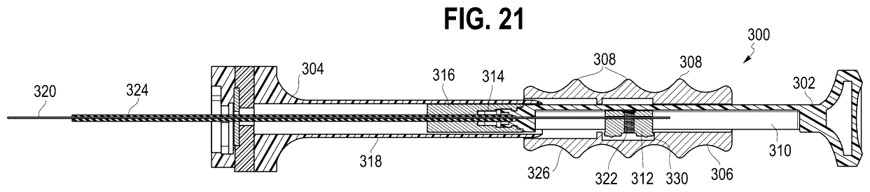

FIG. 21 shows the internal mechanism of the third embodiment. As shown, the handle 300 includes a proximal end 302, a distal end 304, a grip feature 306 having gripping ridges 308, stem 310, insert 312 (which may be brass), sheath grip 314, cap 316, rigid guide 318, drive wire 320, set screw 322, coil spring/sheath 324, and an actuator 326. Sheath grip 314, retained in cap 316, holds sheath/coil spring 324 in place relative to the stem 310. In embodiments, the sheath grip 314 may rotate relative to the cap 316 or alternatively is non-rotatable relative to the cap 316. The cap 316 is held within the rigid guide 318. Again, in embodiments, the cap 316 is rotatable in the rigid guide 318. Alternatively, the cap 316 is non-rotatable in the rigid guide 318.

Set screw 322 is holds the drive wire 320 in place relative to the insert 312 which secures the drive wire 320 inside the actuator 326. The insert 312 is constrained in a cutout 330 in the actuator 326. When a user moves the actuator 326 relative to the stem 310 proximally and distally, this applies force to the drive wire 320, moving the drive wire 320 longitudinally, while the sheath 324 stays in place. This allows the clip (not shown) to open when the actuator 326 is moved distally and close when moved proximally. In addition, cutout 330 in the actuator 326 allows for the insert 312 to rotate with rotation of the stem 310 and apply torque onto the drive wire 320 and rotating the drive wire 320 and clip without rotating the actuator 326. This allows the user to maintain their grip on the actuator 326 while rotating the clip. This also allows rotation of the actuator 326 without rotating the drive wire 320. In embodiments, stem 310 is connected to the cap 316 in a non-rotatable manner. Alternatively, stem 310 is rotatably connected to the cap 316. As may be appreciated, where the stem 310 is connected to the cap 316 and sheath grip 314 in a non-rotatable manner, the sheath 324 also rotates with the drive wire 320.

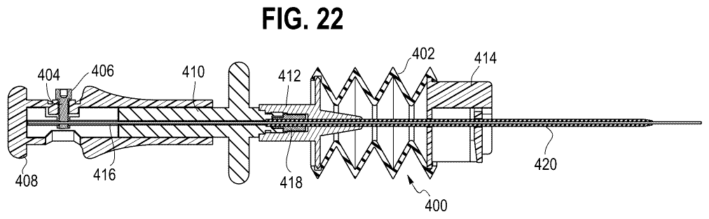

FIG. 22 shows the internal mechanism for the fourth embodiment 400 with accordion 402. This embodiment includes insert 404, screw 406, actuator 408, stem 410, cap mating element 412, scope mount 414, drive wire 416, sheath grip 418 and sheath coil spring 420. Sheath grip 418 holds the sheath/coil spring 420 in place relative to the stem 410. A set screw 406 is used to hold the drive wire 416 in place relative to the insert 404, which secures the wire inside the actuator 408. When the user moves the actuator 408 proximally (see arrow 214 in FIG. 17), it causes the clip to close and eventually deploy. When the user moves the actuator 408 distally (shown by arrow 216 in FIG. 14) it causes the clip to open. The stem 410 and the actuator 408 can rotate relative to cap/mating element 412, accordion 402, and scope mount 414. Turning the actuator 408 or the stem 410 rotates the insert 404, which translates the rotational force to the drive wire 416 while the sheath remains in place. This allows the user to rotate the clip. The accordion 402 can expand/compress, which allows the user to retract/advance the clip. As shown by the arrow 218 in FIG. 17, compression of the accordion advances the clip, whereas expansion of the accordion (as shown by arrow 220 in FIG. 17) retracts the clip.

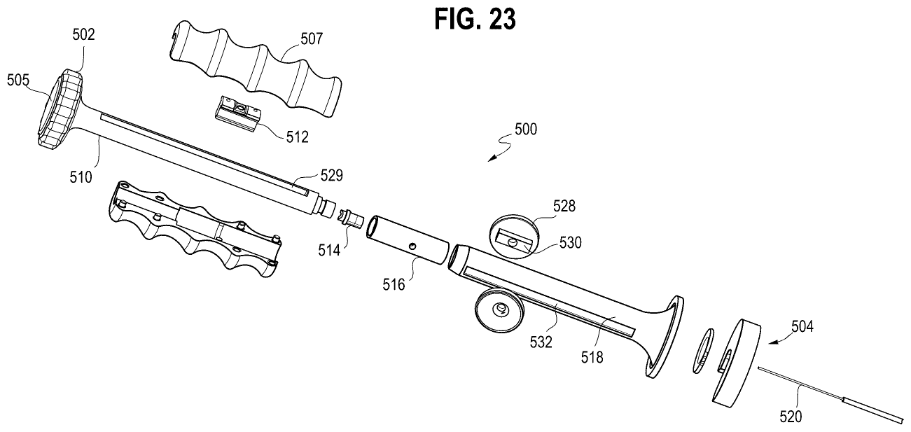

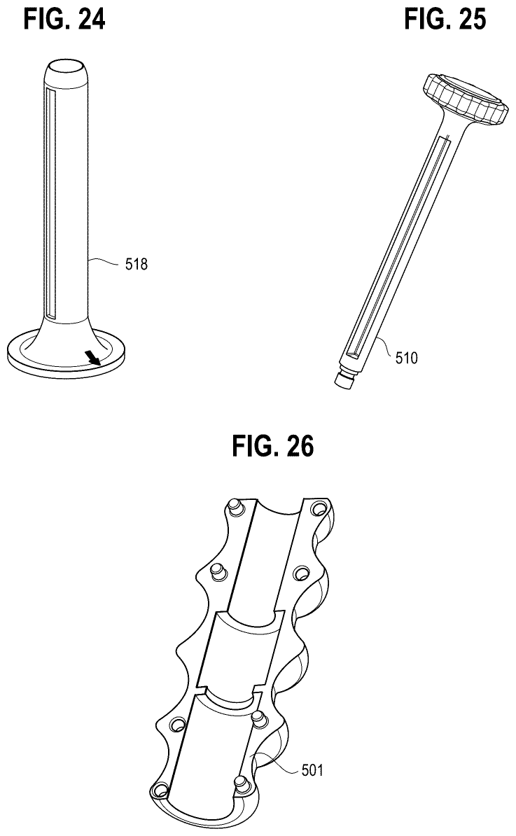

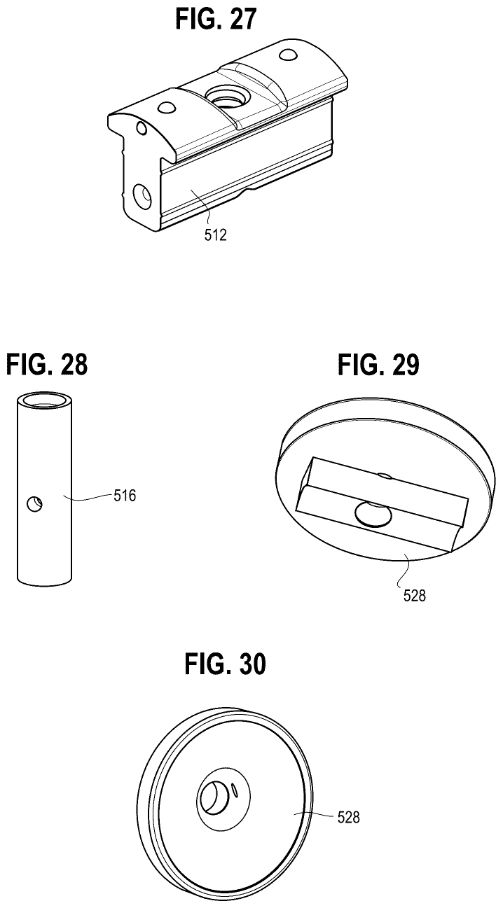

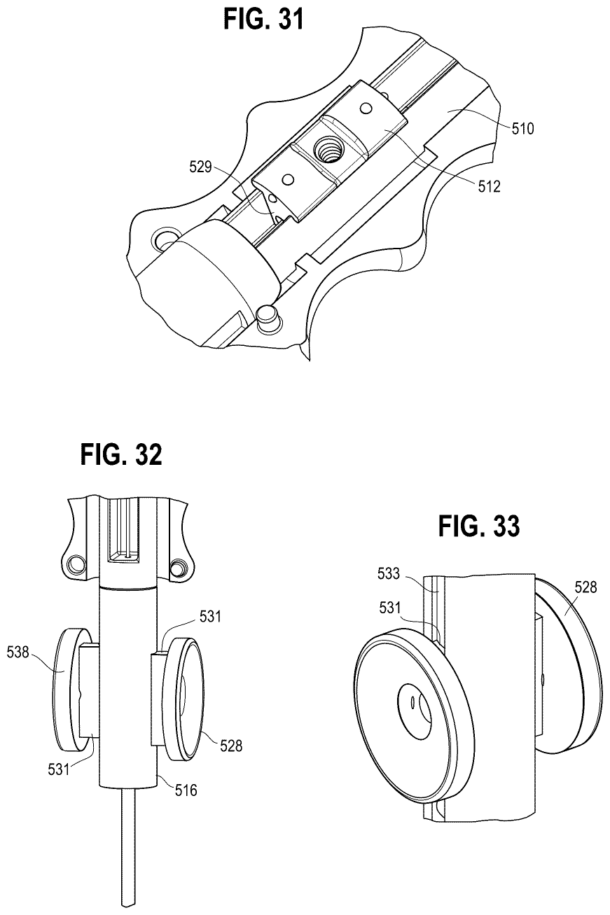

FIG. 23 is an exploded view of the first and second embodiments as shown in FIGS. 19 and 20. As shown, handle 500 has a proximal end 502, a distal end 504, cap 505, grip 507, insert 512, actuator 506, stem 510, sheath grip 514, cap 516, sliders 528 and drive wire 520. FIG. 24 shows rigid guide 518. FIG. 25 shows stem 510. FIG. 26 shows actuator 1 506. FIG. 27 shows insert 512. FIG. 28 shows cap 516. And, FIGS. 29 and 30 show sliders 528. FIG. 31 shows the insert 512. In this embodiment, the insert 512 has a rectangular shape that fits into a rectangular slot 529 of stem 510. FIG. 32 shows the advance/retract sliders 528 screwed into cap 516, which is rotatably seated into the stem 510. FIG. 33 shows the advance/retract sliders 528 with sliding element 531 on the back that seats into slot 532 on rigid guide 518. As shown in FIG. 23, cap 505 is disposed at the proximal end of stem 510. Stem 510 has a slot 529 into which insert 512 is seated. A proximal end (not shown) of drive wire 520 is engaged in insert 512 and held in place by set screw 322 (FIG. 19). Cap 516 is slidably disposed in rigid guide 518. Sliders 528 are mounted onto rigid guide 518 via sliding elements 531 within slot 532. Sliders and sliding elements are engaged with cap 516 which moves within the interior of rigid guide 518 via sliders 528 to advance and retract the clip out of and into the endoscope (not shown).

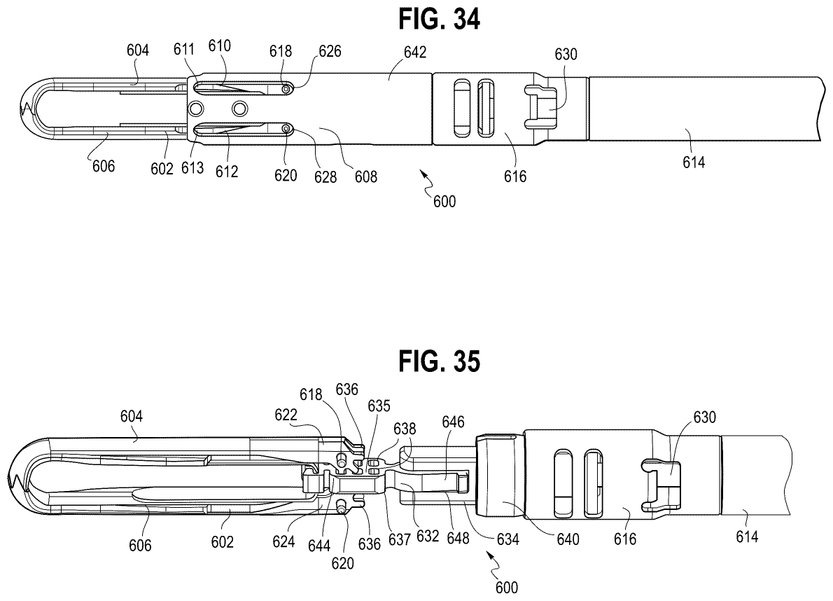

FIGS. 34 through 40 show various view of the clip (distal) end 600 of the system of the present disclosure. In conjunction with the previous figures, the handle is operatively attached at its distal end to an elongate sheath extending from the handle to the clip end of the sheath. The sheath with the clip is inserted into and run through a channel of an endoscope to an end opening of the scope from which the clip may be advanced out of the end of the scope for use and retracted back into the endoscope.

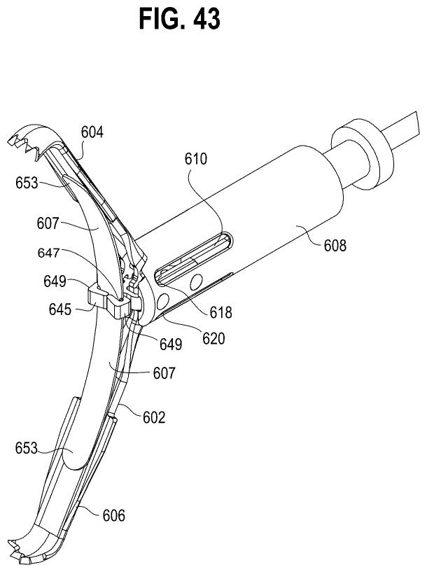

FIG. 34 shows a side view of the clip end 600 of the system. The clip 602 shown in a closed position has a pair of jaws, 604, 606. As shown, the clip 602 is partially enclosed within clip housing 608. Clip housing 608 has two slots 610, 612 (only two shown) on either side of the clip housing 608. The clip housing 608 is shown here attached to the sheath 614 via sheath connector 616, but it is contemplated that the clip housing 608 may be directly connected to the sheath 614. The sheath 614 extends from the housing (or the connector) to the handle (not shown) where it is operatively connected to the stem (not shown) of FIGS. 4 through 24. Pins 618, 620 are disposed within the slots 610, 612 which are slidable along the pins. The pins 618, 620 extend laterally from the proximal ends 622, 624 (shown in FIG. 35) of the jaws 604, 606. As shown in FIG. 34, the pins are disposed at the proximal ends 626, 628 of slots 610, 612. However, when the stem is moved proximally to open the clip, the clip housing 608, the sheath 614, and the housing connector 616 move proximally with the stem. As the clip housing 608 moves proximally, the slots 610, 612 slide along the pins such that the pins are at the distal ends 611, 613 of the slots and the clip is open. Drive wire 630 is operatively attached to the clip 602 and extends through the sheath 614 to the handle. As shown, while the clip housing is disposed over the clip, the clip is closed. As shown in FIG. 43, when the clip housing 608 has been moved proximally from over the clip, the clip is open.

FIG. 35 shows a partial perspective/side view of the clip end 600 of the system without the clip housing 608. As shown, drive wire 630 is connected to a driver 632 which in turn is connected to the proximal ends 622, 624 of the jaws 604, 606. As shown, the driver 632 is connected to the drive wire 630 via a driver/drive wire connector 634. Alternatively, the driver 632 may be directly connected to the drive wire 630. Gear teeth 636 disposed at the proximal ends 622, 624 of the jaws 604, 606 engage slots 638 in the driver 632. Shown here in FIG. 35, each jaw of the pair of jaws is shown here with six gear teeth with three gear teeth each on either side of the proximal ends of the jaws. The six gear teeth of the one jaw engage the slots of a first surface 635 of the driver, while the six gear teach of the other jaw engage the slots in the lower surface 637. As shown, driver 632 has six slots 638. In either case, more or less gear teeth and slots may be utilized. As shown in FIG. 35, clip housing 608 may be connected to the sheath connector 616 via connector 640 which may be disposed within the interior of the proximal end 642 of clip housing 608.

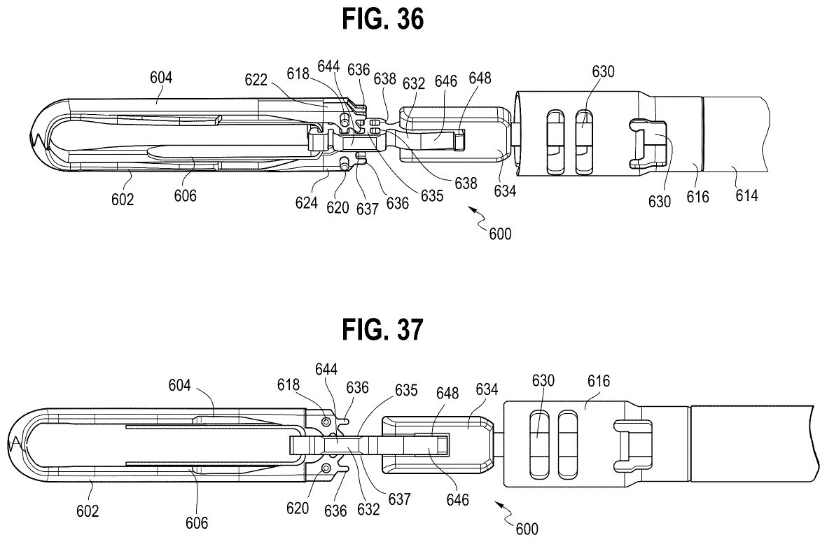

FIG. 36 shows another partial perspective/side view of the clip end 600 of the system. Here the clip end is shown without the clip housing 608 and without connector 640. As shown in both FIGS. 31 and 32, the clip 602 is connected via the distal portion 644 of the driver to the connector 634 via the proximal portion 646 of the driver, which in turn is connected to the drive wire 630. As shown, two teeth of the upper jaw 604 are engaged with upper portion 635 of the distal portion 644 of the driver 632 and two teeth of the lower jaw 606 are engaged with lower portion 637 of the distal portion 644 of the driver 632. A third tooth is shown unengaged. Specifically, the first two sets teeth of the jaws are engaged with the first two holes on the driver 632 and the second two sets of teeth are engaged with the second two holes of the driver 632. When the jaws of the clip are open (not shown here), the teeth pivot and the and the first two sets of teeth pivot out of the first two sets of holes and the third set of teeth pivot into the third set of holes of the driver 632.

FIG. 37 shows a side view of the clip end 600 of FIG. 36 of the system (same reference numerals are applicable here). Specifically shown here, the first two sets teeth of the jaws are engaged with the first two holes on the distal portion of the driver 632 and the second two sets of teeth are engaged with the second two slots 638 on the proximal portion 644 of the driver 632. When the jaws of the clip are open (not shown), the teeth pivot and the and the first two sets of teeth pivot out of the first two sets of holes and the third set of teeth pivot into the third set of holes of the driver 632.

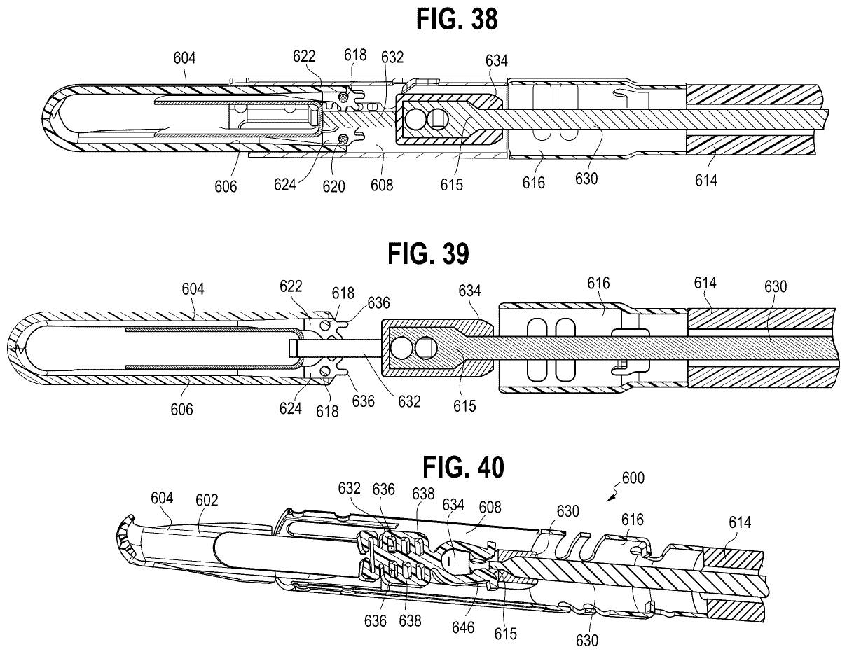

FIGS. 38 and 39 show cross-sections of the clip end 600 of the system of FIGS. 30 through 33. FIG. 38 shows a cross section of the clip end of FIG. 34 with the clip in the closed position. The clip 602 shown in a closed position has a pair of jaws, 604, 606. As shown, the clip is partially enclosed within clip housing 608. The clip housing 608 is shown here attached to the sheath 614 via sheath connector 616, but it is contemplated that the clip housing 608 may be directly connected to the sheath 614. The sheath 614 extends from the housing (or the connector) to the handle (not shown) where it is operatively connected to the stem (not shown). Pins 618, 620 are disposed within the slots 610, 612. The pins 618, 620 extend laterally from the proximal ends 622, 624 of the jaws 604, 606. As shown in FIG. 34, the pins are disposed at the proximal ends 626, 628 of slots 610, 612. Drive wire 630 is operatively attached to the clip 602 and extends through the sheath 614 to the handle. As shown, while the housing is disposed over the clip, the clip is closed. The distal end 615 of the drive wire 630 is engaged with driver/drive wire connector 634, which in turn engages the proximal portion 646 of the driver 632. As shown, the distal end 615 of the drive wire 630 is fitted within connector 634 FIG. 39 shows the view of the clip end of FIG. 38 without the housing.

FIG. 40 shows a perspective cross-section of the clip end 600. A single jaw 604 of the clip 602 is shown. As shown, clip housing 608 is at least partially disposed over the clip 602 and the clip is in a closed position. The proximal portion 646 of driver 632 is disposed within, for example clamped or otherwise secured within, the proximal portion 646 of driver 632. As shown in FIGS. 35 through 37, the proximal portion of the driver extends into slot 648 of the connector 634 to engage and connect with the distal end 615 of the drive wire 630. As shown, driver 632 has a proximal portion 644 having slots 638 which are engaged with gear teeth 636.

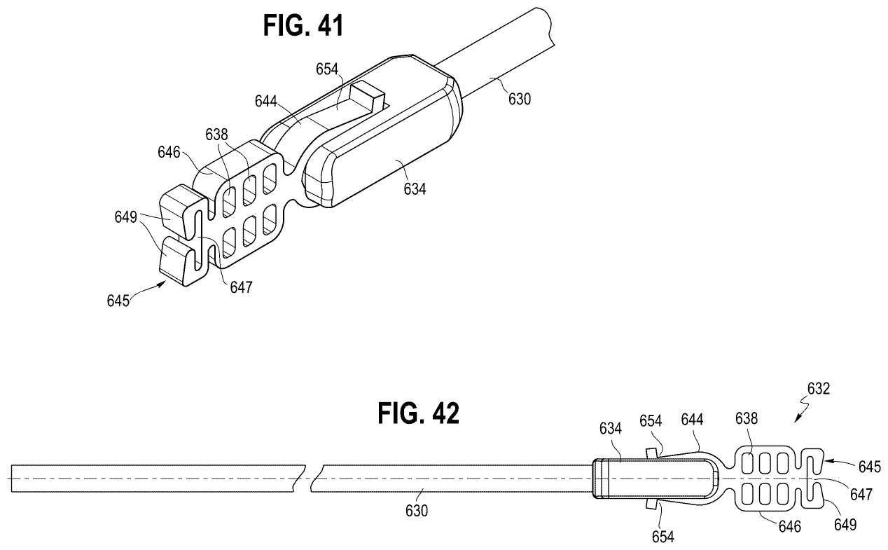

FIG. 41 shows a perspective view of the attachment between the driver 632, the driver/driver wire connector 634 and the drive wire 630. FIG. 42 shows a side view of the attachment of the driver 632 to the distal end 615 of the drive wire 630. As shown, the proximal portion 644 of the driver 632 has a plurality of holes or slots 638 with which the gear teeth (not shown) engage when the clip is opened and closed. The driver has a proximal portion 644, distal portion 646 and a distal end 645. The distal end has pocket 647 and flanges 649, as will be explained with regard to FIG. 43. As shown proximal portion 644 engages with the driver/drive wire connector 634 and hence the drive wire 630.

FIG. 43 shows the clip 602 in the open position with the clip housing 608 withdrawn from over the jaws of the clip 602. As shown, the distal end 645 of the driver 632 receives the biasing strip 607 within the pocket 647. The flanges 649 are bent inwardly and proximally as shown to firmly engage the biasing strip 607 and fix it to the driver 632. The biasing strip 607 is preferably a thin strip formed from a sheet of resilient material, and more preferably a metal strip, e.g., formed of stainless steel, nitinol or other super elastic alloy that is biocompatible. Accordingly, it will be recognized that as the stem is moved distally cause the jaws 604, 606 to close, the biasing strip 607 will be forced into a V-shape or U-shape, as shown in the previous figures. That is, the biasing strip 607 has a substantially straight shape, or somewhat curved shape as shown in FIG. 43 in its natural, unbiased, configuration, and when bent into the V-shape or U-shape it exerts a radially outward force on the jaws 604, 606. This biasing force provides the jaws 604, 606 with smooth rotation and transition between the open and closed positions. It will also be recognized that the biasing strip 607 could also have its original, unbiased position formed as a V-shape or a U-shape, and be affixed to the jaws 604,606 such that it exerts a radially inward biasing force. The free ends 653 of the biasing strip 607 simply press against the jaws 604, 606 but may not be fixed or rigidly attached thereto. Suitable configurations for a biasing mechanism are described and disclosed in U.S. Pat. No. 11,576,682, in particular FIGS. 22 through 23 and the accompanying text, the contents of which are incorporated by reference herein in their entirety.

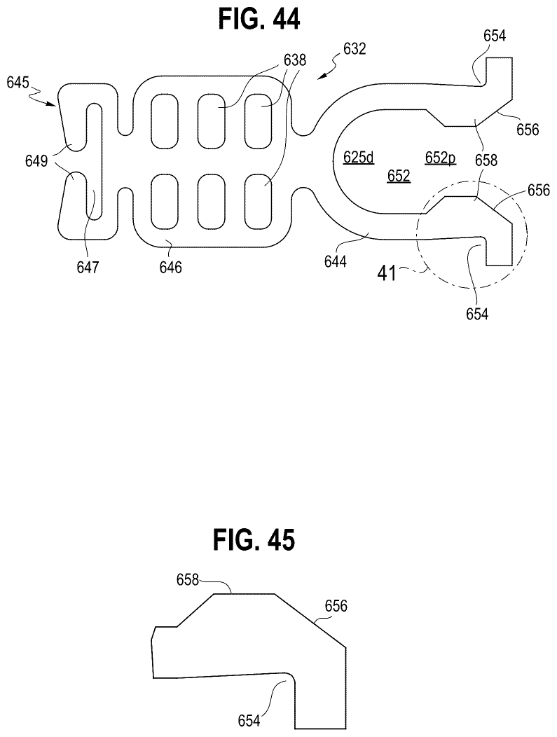

FIG. 44 shows an exemplary driver 632. As shown, driver 632 has a proximal portion 644, a distal portion 646 and a distal end 645. As described above, the distal end 645 has a pocket 647 and flanges 649 that are bent inwardly and proximally. The driver 632 generally includes a socket 652 formed by two locking tabs 654. As shown, proximal portion of the locking tabs may define slanted shoulders 656 which slope laterally outwardly for engagement with the driver/driver wire connector 634. The locking tabs also may include inner projections 658 which project laterally inwardly and separate the socket 652 into a distal portion 652d and a proximal portion 652p. FIG. 45 is an enlargement of the locking tabs.

The handle of the present disclosure allows advancement and retraction of the clip, rotation of the clip, opening and closing of the clip, and deployment of the clip by a physician using a single hand without any assistance from another clinician/technician. With any of the three embodiments (the rigid sheath, the rigid guide or the accordion mount, the physician need only place his/her hand on the grip and thumb on the cap to open, close, rotate, advance, retract and deploy the clip. The handle permits greater than 90° rotation with the one-handled manipulation. The handle also allows for advancement of the clip from 0 to 10 cm, and preferably 0 to 6 cm distal to the scope. Although each of the embodiments described refer to the stem/cap being moved relative to the grip to open, close and release the clip, movement of the grip relative to the stem/cap are also contemplated to open, close and release the clip.

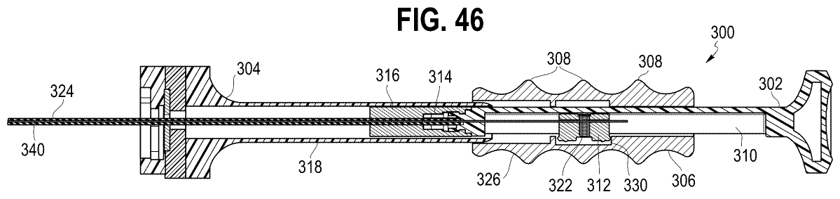

As noted above, the novel single hand operated handle may alternatively be used with other elongate members instead of the drive wire and devices instead of the hemo-clip. In further embodiments, the drive wire may be substituted with another elongate member, such as a needle, trocar, or cannula. FIG. 46 illustrates an embodiment where the drive wire and hemo-clip of the above embodiments is replaced with another elongate member, a needle 340. As the actuator 306 is moved distally towards the endoscope, the needle 340 is advanced out of the sheath 320. Further, the needle 340 is rotatable upon rotation of the stem 310 relative to the actuator 306. Or as illustrated in the embodiment of FIG. 22, the needle 340 is extendable out of the sheath 420 when the actuator 408 is moved distally, towards the scope, and rotatable upon rotation of the actuator 408 or, alternatively, rotation of both the actuator 408 and stem 410.

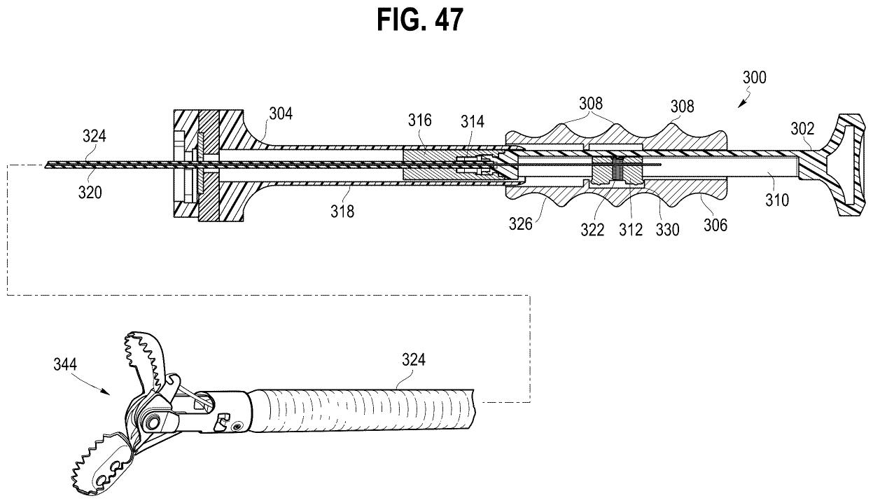

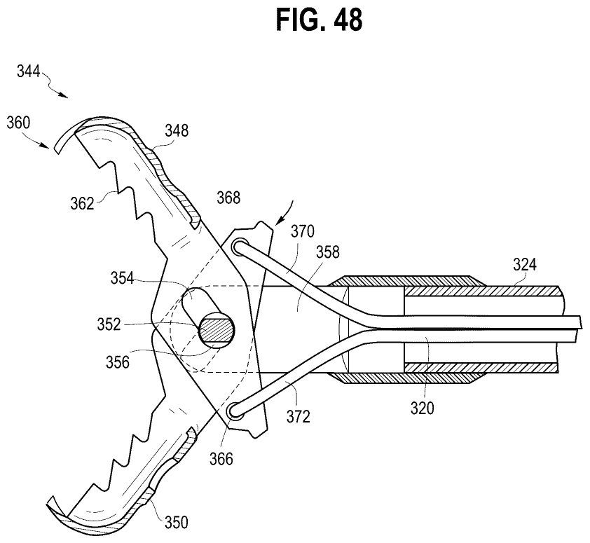

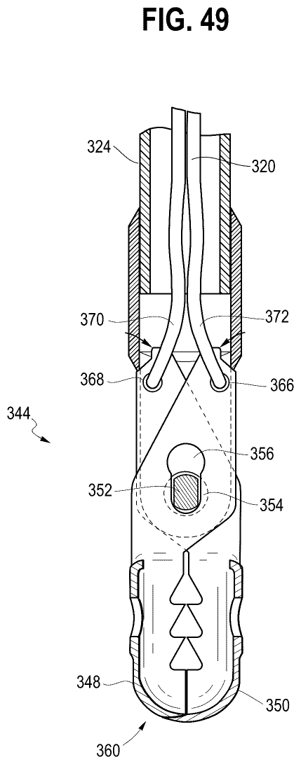

Further, instead of a hemo-clip, a snare or forceps may be used and actuated by the drive wire. FIG. 47 illustrates an embodiment wherein the hemo-clip is replaced with forceps 344, which open and close upon movement of the drive wire 320 or other elongate member. FIG. 48 illustrates the forceps 344 in a first, open configuration and FIG. 49 illustrates the forceps 344 in a second, closed configuration. With reference to FIGS. 47 through 49, the forceps include a pair of jaws 348, 350 that are mounted on a pin 352. The jaws 348, 350 each define a slot 354 including a circular opening 356 at the proximal end of the slot 354 allowing the jaws 348, 350 to translate longitudinally and rotate relative to each other on the pin 352. The pin 352 is mounted to a housing 356 and exhibits an oblong or elliptical shape. The housing 358 is connected to the sheath 324.

Each jaw 348, 350 includes a distal end 360, which may include serrated teeth 362 as illustrated. Further, at the proximal end 364 of each jaw 348, 350, the jaws 348, 350 include a connection point 366, 368 which rotatably receives a connection member 370, 372. The connection members 370, 372 are rotatably connected to the drive wire 320. Movement of the drive wire 320 distally relative to the sheath 324 by moving the actuator 306 distally pushes the jaws 348, 350 out relative to the housing 358. The slots 354 slide distally over the pin 352 until the pin 352 is located in the opening 356. Continued distal motion of the drive wire 320 causes the connection members 370, 372 to rotate the jaws 348, 350 relative to each other around the pin 352 and open as illustrated in FIG. 48. Movement of the drive wire 320 proximally relative to the sheath 324 by moving the actuator 306 proximally causes the connection members 370, 372 to rotate the jaws 348, 350 around the pin 521 and close. Further proximal movement of the drive wire 320 causes the slots 354 to slide longitudinally over the pin 352 and maintain the jaw 348, 350 in the closed position as illustrated in FIG. 49.

While various embodiments of the disclosure have been described, the disclosure is not to be restricted except in light of the attached claims and their equivalents. Moreover, the advantages described herein are not necessarily the only advantages of the disclosure and it is not necessarily expected that every embodiment of the disclosure will achieve all of the advantages described. Each of the embodiments described further may include all or only a subset of the features described herein.

What is claimed is:

1. A physician controlled single hand operated handle system, comprising:
a handle, the handle comprising:
an actuator;
a stem disposed within the actuator and longitudinally movable within the actuator;
a sheath attached to the stem; and
an elongate member extending through the sheath, wherein the elongate member is longitudinally constrained by the actuator and rotatably constrained by the stem.

2. The system of claim 1, wherein the stem includes a proximal end, and the stem includes a wheel at the proximal end, wherein the wheel is rotatable with the stem.

3. The system of claim 1, wherein the elongate member is held in an insert, and the insert is longitudinally constrained in a cutout in the actuator.

4. The system of claim 3, wherein the elongate member is held in the insert with a set screw.

5. The system of claim 3, wherein the insert is rotatably constrained by the stem, and the elongate member and stem are rotatable relative to the actuator.

6. The system of claim 3, wherein the insert is rotatably constrained by the actuator.

7. The system of claim 1, wherein the sheath is connected to a sheath grip and the sheath grip holds the sheath relative to the stem.

8. The system of claim 7, wherein the sheath grip is non-rotatably retained by a cap in a rigid mount and the elongate member and sheath pass through the rigid mount.

9. The system of claim 8, wherein the cap is non-rotatably secured to the stem.

10. The system of claim 8, further comprising a slider extending through a slot defined in the rigid mount, wherein the slider is connected to the cap and the slider is configured to move the cap longitudinally in a rigid mount.

11. The system of claim 7, wherein the sheath grip is non-rotatably retained by a cap and the cap is connected to an accordion and the elongate member and sheath pass through the accordion.

12. The system of claim 11, wherein the accordion includes a scope mount at a distal end of the handle.

13. The system of claim 1, wherein a distal end of the handle comprises a scope mount configured to engage with a mount of an endoscope.

14. The system of claim 13, wherein the scope mount includes a first magnet received in a first channel defined in the scope mount and the system further comprising a port mount magnetically couple-able to the scope mount, wherein the port mount includes a second magnet received in a second channel defined in the port mount.

15. The system of claim 14, wherein the port mount includes a seal.

16. The system of claim 1, wherein the elongate member has a proximal end and a distal end, and wherein the proximal end is operatively coupled to the actuator and the distal end is operatively coupled to a clip.

17. The system of claim 16, wherein the clip exhibits an open configuration and a closed configuration wherein proximal longitudinal movement of the stem relative to the actuator opens the clip, and wherein a first distal longitudinal movement of the stem relative to the actuator closes the clip.

18. The system of claim 17, wherein a second distal longitudinal movement of the stem relative to the actuator releases the clip from the system.

19. The system of claim 17, wherein the clip includes a pair of jaws and the system further comprises a clip housing configured to receive the pair of jaws.

20. The system of claim 1, wherein the elongate member is at least one of a needle and a trocar.
